(19) United States
(12) Patent Application Publication
(10) Pub. No.: US 2010/0086567 A1
(43) Pub. Date: Apr. 8, 2010

(54) Botulinum toxin therapy for prevention of anastamotic thrombosis in free tissue transfer

(76) Inventor: Mark Warren Clemens, II, Woodridge, VA (US)

Correspondence Address: OBER / KALER;A Professional Corporation, 120 East Baltimore Street, Baltimore, MD, 21202-1643, US

(21) Appl. No.: 12/587,110

(22) Filed: Oct. 1, 2009

Related U.S. Application Data
(60) Provisional application No. 61/194,864, filed on Oct. 1, 2008.

(51) Int. Cl. A61K 39/08; A61P 7/00

(52) U.S. Cl. 424/239.1

(57) ABSTRACT

An effective, long lasting, non-systemic drug administration protocol for preventing vascular vasospasm and vessel thrombosis in free tissue transplantation. In a free flap procedure, after the recipient vein and artery (vascular pedicle) are divided, the recipient artery and vein are pretreated with a local injection of botulinum toxin. The vein and artery from the flap (vascular pedicle) are anastomosed (connected) to the vein and artery identified using microsurgery. The free flap is sutured to the prepared area, and the donor site is sutured closed. Then, a relatively long term, non-systemic local administration neurotoxin administration protocol is effected in conjunction with a periodic monitoring program to ensure the blood vessels remain patent (i.e. the vessels have good blood flow). The botulinum post-treatment alone or in combination with the botulinum pretreatment can proactively prevent vascular vasospasm and vessel thrombosis after the free tissue transplantation.

### CROSS-REFERENCE TO RELATED APPLICATION(S)

The present application derives priority from U.S. provisional application Ser. No. 61/194,864 filed Oct. 1, 2008.

### BACKGROUND OF THE INVENTION

1. Field of the Invention

The present invention relates generally to free tissue transfer procedure and, more specifically, to a method for decreasing the incidence of vascular thrombosis and free tissue transplant loss by administration of a locally acting neurotoxin to a patient.

2. Description of the Background

Free tissue transfer is a surgical reconstructive procedure using microsurgery. Free tissue transfer or transplantation is used widely for the reconstruction of complex defects and disorders throughout the body, including cancer reconstructions of the breast, upper and lower extremities, and the head and neck. A region of “donor” tissue is selected that can be isolated on a feeding artery and vein. The donor tissue is usually a composite of several tissue types (e.g., skin, muscle, fat, bone). The donor tissue is transferred to recipient vessels such as the external jugular (EJ) vein and internal jugular (IJ). Free tissue transfer can be moved to any part of the body, not just the EJ or IJ. Any artery or vein may act as recipient vessels. This allows for reconstructions of virtually any part of the body. It is used frequently for head and neck surgery, lower extremity reconstruction, breast reconstruction, and most any type of replantation such as fingers and hands. Since the introduction of free tissue transplantation thirty years ago, the success rate has improved substantially and currently is estimated at 95-98% among experienced surgeons. The success of a free tissue transfer (or “free flap”) is dependent upon a variety of factors related to the patient, the operative technique, and the postoperative management. Patient variables include advanced age, nutritional status, tobacco usage, and presence of underlying comorbidities (eg, diabetes mellitus, cardiopulmonary disease, peripheral vascular disease). The most common cause for failure of a free flap remains vascular thrombosis (closure by clotting), at the anastamosis (site where the arteries and veins are sewn together). The prevalence of thrombosis is evenly distributed among artery, vein, and both. Attempts to salvage a failing flap decrease precipitously to only 15 percent when both vessels (artery and vein) thrombose. Factors contributing to a difficult anastamosis include vasospasm, trauma to the zone of injury, radiation, scar, and infection. Thrombosis typically occurs within the first 2 days in 80% of patients. Existing techniques used to prevent vasospasm and thrombosis include abstinence from vasopressors (medicines that cause vessel constriction) during procedures, limited intraoperative manipulation of vessels, serosal stripping of vessels (removal of nerves), and post-operative warming protocols. All of these mechanisms are sympathetically mediated (controlled by nerves).

Botulinum toxin is a well-known muscle relaxant. The anaerobic, gram positive bacterium Clostridium botulinum produces a potent polypeptide neurotoxin, botulinum toxin, which causes a neuroparalytic illness in humans and animals referred to as botulism. The spores of Clostridium botulinum are found in soil and can grow in improperly sterilized and sealed food containers of home based canneries, which are the cause of many of the cases of botulism. The effects of botulism typically appear 18 to 36 hours after eating the foodstuffs infected with a Clostridium botulinum culture or spores. The botulinum toxin can apparently pass unattenuated through the lining of the gut and attack peripheral motor neurons. Symptoms of botulinum toxin intoxication can progress from difficulty walking, swallowing, and speaking to paralysis of the respiratory muscles and death.

Botulinum toxin type A is the most lethal natural biological agent known to man. About 50 picograms of a commercially available botulinum toxin type A (purified neurotoxin complex) is a LD₅₀ in mice (i.e. 1 unit). Interestingly, on a molar basis, botulinum toxin type A is about 1.8 billion times more lethal than diphtheria, about 600 million times more lethal than sodium cyanide, about 30 million times more lethal than cobra toxin and about 12 million times more lethal than cholera. Singh, Critical Aspects of Bacterial Protein Toxins, pages 63-84 (chapter 4) of Natural Toxins II, edited by B. R. Singh et al., Plenum Press, New York (1976) (where the stated LD₅₀ of botulinum toxin type A of 0.3 ng equals 1 U is corrected for the fact that about 0.05 ng of BOTOX™ equals 1 unit). One unit (U) of botulinum toxin is defined as the LD₅₀ upon intraperitoneal injection into female Swiss Webster mice weighing 18 to 20 grams each.

Seven immunologically distinct botulinum neurotoxins have been characterized, these being respectively botulinum neurotoxin serotypes A, B, C₁, D, E, F and G each of which is distinguished by neutralization with type-specific antibodies. The different serotypes of botulinum toxin vary in the animal species that they affect and in the severity and duration of the paralysis they evoke. For example, it has been determined that botulinum toxin type A is 500 times more potent, as measured by the rate of paralysis produced in the rat, than is botulinum toxin type B. Additionally, botulinum toxin type B has been determined to be non-toxic in primates at a dose of 480 U/kg which is about 12 times the primate LD₅₀ for botulinum toxin type A. Botulinum toxin apparently binds with high affinity to cholinergic motor neurons, is translocated into the neuron and blocks the release of acetylcholine.

Despite the ominous toxic effect, researchers discovered in the 1950s that injecting minute quantities of botulinum toxin type A decreased muscle activity by blocking the release of acetylcholine at the neuromuscular junction, thereby rendering the muscle unable to contract for a period 3 to 4 months. Botulinum toxins have since been used in clinical settings for the treatment of neuromuscular disorders characterized by hyperactive skeletal muscles. Botulinum toxin was originally used to treat ‘crossed eyes’ (strabismus) and ‘uncontrollable blinking’ (blepharospasm), and Allergan obtained FDA approval in 1989 and renamed the drug Botox™. The use of Botulinum toxin for cosmetic purposes occurred in the early 2000 when it was found to reduce frown lines, and this led to another round of clinical trials and subsequent FDA approval for cosmetic use in April 2002. Now there are 4.6 million Botox™ injection procedures in the United States every year. Besides its cosmetic application, Botox™ has also been used in the treatment of cervical dystonia (a neuromuscular disorder involving the head and neck), blepharospasm (involuntary blinking), severe primary axillary hyperhidrosis (excessive sweating), achalasia (failure of the lower esophageal sphincter to relax), migraine and other headache disorders, incontinence, Parkinson's disease, cerebral palsy, and wound healing. Botulinum toxin type A has been approved by the U.S. Food and Drug Administration for the treatment of blepharospasm, strabismus and hemifacial spasm.

Botulinum toxin type A can be obtained by establishing and growing cultures of Clostridium botulinum in a fermenter and then harvesting and purifying the fermented mixture in accordance with known procedures. All the botulinum toxin serotypes are initially synthesized as inactive single chain proteins which must be cleaved or nicked by proteases to become neuroactive. The bacterial strains that make botulinum toxin serotypes A and G possess endogenous proteases and serotypes A and G can therefore be recovered from bacterial cultures in predominantly their active form. In contrast, botulinum toxin serotypes C₁, D and E are synthesized by nonproteolytic strains and are therefore typically unactivated when recovered from culture. Serotypes B and F are produced by both proteolytic and nonproteolytic strains and therefore can be recovered in either the active or inactive form. However, even the proteolytic strains that produce, for example, the botulinum toxin type B serotype only cleave a portion of the toxin produced. The exact proportion of nicked to unnicked molecules depends on the length of incubation and the temperature of the culture. Therefore, a certain percentage of any preparation of, for example, the botulinum toxin type B toxin is likely to be inactive, possibly accounting for the known significantly lower potency of botulinum toxin type B as compared to botulinum toxin type A. Additionally, it is known that botulinum toxin type B has, upon intramuscular injection, a shorter duration of activity and is also less potent than botulinum toxin type A at the same dose level. The presence of inactive botulinum toxin molecules in a clinical preparation will contribute to the overall protein load of the preparation, which has been linked to increased antigenicity, without contributing to its clinical efficacy.

High quality crystalline botulinum toxin type A can be produced from the Hall A strain of Clostridium botulinum with characteristics of 7 U/mg, an A₂₆₀/A₂₇₈ of less than 0.60 and a distinct pattern of banding on gel electrophoresis. The known Shantz process can be used to obtain crystalline botulinum toxin type A, as set forth in Shantz, E. J., et al, Properties and Use of Botulinum Toxin And Other Microbial Neurotoxins In Medicine, Microbiol Rev. 56: 80-99 (1992). Generally, the botulinum toxin type A complex can be isolated and purified from an anaerobic fermentation by cultivating Clostridium botulinum type A in a suitable medium. The known process can also be used, upon separation out of the non-toxin proteins, to obtain pure botulinum toxins, such as for example: purified botulinum toxin type A with an approximately 150 kD molecular weight with a specific potency of 1-2×10⁸ LD₅₀ U/mg or greater; purified botulinum toxin type B with an approximately 156 kD molecular weight with a specific potency of 1-2×10⁸ LD₅₀ U/mg or greater, and; purified botulinum toxin type F with an approximately 155 kD molecular weight with a specific potency of 1-2×10⁷ LD₅₀ U/mg or greater. Non-type A botulinum toxin serotypes apparently have a lower potency and/or a shorter duration of activity as compared to botulinum toxin type A.

Already prepared and purified botulinum toxins and toxin complexes can be obtained from List Biological Laboratories, Inc., Campbell, Calif.; the Centre for Applied Microbiology and Research, Porton Down, U.K.; Wako (Osaka, Japan), as well as from Sigma Chemicals of St Louis, Miss. However, pure botulinum toxin is so labile that it is generally not used to prepare a pharmaceutical composition. Furthermore, the botulinum toxin complexes, such the toxin type A complex are also extremely susceptible to denaturation due to surface denaturation, heat, and alkaline conditions. Inactivated toxin forms toxoid proteins which may be immunogenic. The resulting antibodies can render a patient refractory to toxin injection. The commercially available botulinum toxin containing pharmaceutical composition Botox™ is available from Allergan, Inc., of Irvine, Calif. Botox™ consists of a purified botulinum toxin type A complex, albumin and sodium chloride packaged in sterile, vacuum-dried form. The botulinum toxin type A is made from a culture of the Hall strain of Clostridium botulinum grown in a medium containing N-Z amine and yeast extract. The botulinum toxin type A complex is purified from the culture solution by a series of acid precipitations to a crystalline complex consisting of the active high molecular weight toxin protein and an associated hemagglutinin protein. The crystalline complex is re-dissolved in a solution containing saline and albumin and sterile filtered (0.2 microns) prior to vacuum-drying. Each vial of Botox™ contains about 100 units (U) of Clostridium botulinum toxin type A purified neurotoxin complex, 0.5 milligrams of human serum albumin and 0.9 milligrams of sodium chloride in a sterile, vacuum-dried form without a preservative. Thus, one unit of Botox™ contains about 50 picograms of botulinum toxin type A complex. The vacuum-dried product is stored in a freezer at or below 5 degrees C. Botox™ can then be reconstituted with sterile, non-preserved saline (0.9% Sodium Chloride) prior to intramuscular injection. Since Botox™ can be denatured by bubbling or similar violent agitation, the diluent is gently injected into the vial. The diluted Botox™ should be administered within four hours after reconstitution. During this time period, reconstituted Botox™ is stored in a refrigerator (20 to 8 degrees C.). Reconstituted Botox™ is clear, colorless and free of particulate matter.

Clinical effects of peripheral intramuscular botulinum toxin type A are usually seen within one week of injection. The typical duration of symptomatic relief from a single intramuscular injection of botulinum toxin type A averages about three months. In vitro studies have indicated that botulinum toxin inhibits potassium cation induced release of both acetylcholine and norepinephrine from primary cell cultures of brainstem tissue. Additionally, it has been reported that botulinum toxin inhibits the evoked release of both glycine and glutamate in primary cultures of spinal cord neurons and that in brain synaptosome preparations botulinum toxin inhibits the release of each of the neurotransmitters acetylcholine, dopamine, norepinephrine, CGRP and glutamate. As with enzymes generally, the biological activities of the botulinum toxins (which are intracellular peptidases) is dependant, at least in part, upon their three dimensional conformation. Thus, botulinum toxin type A is detoxified by heat, various chemicals surface stretching and surface drying. Additionally, it is known that dilution of the toxin complex obtained by the known culturing, fermentation and purification to the much, much lower toxin concentrations used for pharmaceutical composition formulation results in rapid detoxification of the toxin unless a suitable stabilizing agent is present. Dilution of the toxin from milligram quantities to a solution containing nanograms per milliliter presents significant difficulties because of the rapid loss of specific toxicity upon such great dilution. Since the toxin may be used months or years after the toxin containing pharmaceutical composition is formulated, the toxin must be formulated with a stabilizing agent, such as albumin.

It has been reported that botulinum toxin type A has been used in clinical settings as follows:

(1) about 75-125 units of Botox™ per intramuscular injection (multiple muscles) to treat cervical dystonia;

(2) 5-10 units of Botox™ per intramuscular injection to treat glabellar lines (brow furrows) (5 units injected intramuscularly into the procerus muscle and 10 units injected intramuscularly into each corrugator supercilii muscle);

(3) about 30-80 units of Botox™ to treat constipation by intrasphincter injection of the puborectalis muscle;

(4) about 1-5 units per muscle of intramuscularly injected Botox™ to treat blepharospasm by injecting the lateral pre-tarsal orbicularis oculi muscle of the upper lid and the lateral pre-tarsal orbicularis oculi of the lower lid.

(5) to treat strabismus, extraocular muscles have been injected intramuscularly with between about 1-5 units of Botox™, the amount injected varying based upon both the size of the muscle to be injected and the extent of muscle paralysis desired (i.e. amount of diopter correction desired).

(6) to treat upper limb spasticity following stroke by intramuscular injections of Botox™ into five different upper limb flexor muscles, as follows:
(a) flexor digitorum profundus: 7.5 U to 30 U
(b) flexor digitorum sublimus: 7.5 U to 30 U
(c) flexor carpi ulnaris: 10 U to 40 U
(d) flexor carpi radialis: 15 U to 60 U
(e) biceps brachii: 50 U to 200 U. Each of the five indicated muscles has been injected at the same treatment session, so that the patient receives from 90 U to 360 U of upper limb flexor muscle Botox™ by intramuscular injection at each treatment session.

(7) to treat migraine, pericranial injected (injected symmetrically into glabellar, frontalis and temporalis muscles) injection of 25 U of Botox™ has showed significant benefit as a prophylactic treatment of migraine compared to vehicle as measured by decreased measures of migraine frequency, maximal severity, associated vomiting and acute medication use over the three month period following the 25 U injection.

Many of the foregoing applications for Botox™ have been patented. For example, botulinum toxin to treat skin wounds (U.S. Pat. No. 6,447,787), autonomic nerve dysfunctions (U.S. Pat. No. 5,766,605), tension headache, (U.S. Pat. No. 6,458,365), migraine headache (U.S. Pat. No. 5,714,468), post-operative pain and visceral pain (U.S. Pat. No. 6,464,986 and EP1334729), hair growth and hair retention (U.S. Pat. No. 6,299,893), psoriasis and dermatitis (U.S. Pat. No. 5,670,484), injured muscles (U.S. Pat. No. 6,423,319) various cancers (U.S. Pat. No. 6,139,845), smooth muscle disorders (U.S. Pat. No. 5,437,291), nerve entrapment syndrome (20030224019), acne (WO 03/011333) and neurogenic inflammation (U.S. Pat. No. 6,063,768). Various release mechanisms are covered as well, such as controlled release toxin implants (U.S. Pat. Nos. 6,306,423 and 6,312,708) and transdermals (20040009180).

Despite the prevalent applications, the mechanism is only partially understood. Botulinum toxin blocks the release of acetylcholine. The neurotransmitter acetylcholine is secreted by neurons in many areas of the brain, but specifically by the large pyramidal cells of the motor cortex, by several different neurons in the basal ganglia, by the motor neurons that innervate the skeletal muscles, by the preganglionic neurons of the autonomic nervous system (both sympathetic and parasympathetic), by the postganglionic neurons of the parasympathetic nervous system, and by some of the postganglionic neurons of the sympathetic nervous system. Essentially, only the postganglionic sympathetic nerve fibers to the sweat glands, the piloerector muscles and a few blood vessels are cholinergic as most of the postganglionic neurons of the sympathetic nervous system secret the neurotransmitter norepinephine. Acetylcholine activates two types of receptors, muscarinic and nicotinic receptors. The muscarinic receptors are found in all effector cells stimulated by the postganglionic neurons of the parasympathetic nervous system, as well as in those stimulated by the postganglionic cholinergic neurons of the sympathetic nervous system. The nicotinic receptors are found in the synapses between the preganglionic and postganglionic neurons of both the sympathetic and parasympathetic. The nicotinic receptors are also present in many membranes of skeletal muscle fibers at the neuromuscular junction. In most instances acetylcholine has an excitatory effect. However, acetylcholine is known to have inhibitory effects at some of the peripheral parasympathetic nerve endings, such as inhibition of heart rate by the vagal nerve. A wide variety of non-neuronal secretory cells, such as, adrenal medulla (as well as the PC12 cell line) and pancreatic islet cells release catecholamines and thyroid hormone, respectively, from large dense-core vesicles. Botulinum toxin inhibits the release of both types of compounds from both types of cells in vitro, permeabilized (as by electroporation) or by direct injection of the toxin into the denervated cell. Botulinum toxin is also known to block release of the neurotransmitter glutamate from cortical synaptosomes cell cultures. Botulinum toxin A has also been shown to prevent the release of norepinephrine at the neuromuscular junction and subsequently prevented sympathetic vasoconstriction of vascular smooth muscle. Morris et al., Differential Inhibition By Botulinum Neurotoxin A Of Cotransmitters Released From Autonomic Vasodilator Neurons, Am J Physiol

Heart Circ. Physiol 281: H2124-H2132, 2001. Norepinephrine vesicles were blocked from release at the neuromuscular junction. Vasoconstriction is also dependent upon the recruitment of specific alpha 2 receptors (alpha 2c) involving a phospholipase D pathway that is completely blocked by botulinum toxin. Jinsi-Parimoo et al., Reconstitution Of Alpha2d-Adrenergic Receptor Coupling To Phospholipase D In a PC12 Cell Lysate, J Biol. Chem. 272: 14556-14561, 1997. Consequently, it has been theorized that botulinum toxin A can inhibit vasospasm by two mechanisms: by blocking sympathetically mediated cold-induced vasoconstriction and by preventing recruitment of alpha 2 receptors in vascular smooth muscle. Van Beek et al., Management Of Vasospastic Disorders With Botulinum Toxin A, Plast. Reconstr. Surg 119: 217-226, 2007. Further studies have shown increased blood flow, muscle perfusion, and glucose uptake after the administration of botulinum toxin. Matic et al., The Effects Of Botulinum Toxin Type A On Muscle Blood Perfusion And Metabolism, Plast. Reconstr. Surg 120: 1823-1833, 2007. Botulinum toxin A shows an onset between 3 to 5 days and lasts several weeks to months. Muscle functional recovery occurs by a mechanism of sprouting of new nerve terminals.

The present inventor has noted the relevance of these effects and the potential for the mechanism in the context of free tissue transfer, where preventing vasoconstriction, and maintaining increased vessel diameter and blood flow at the anastamosis site (site where the arteries and veins are sewn together) could help prevent vascular vasospasm and thrombosis (closure by clotting) and increase the success rate of free tissue transfers. What is provided, therefore, is an effective, long lasting, non-systemic drug administration, therapeutic drug and method for preventing vascular vasospasm and vessel thrombosis in free tissue transplantation.

### SUMMARY OF THE INVENTION

It is, therefore, the primary object of the present invention to provide an effective, long lasting, non-systemic neurotoxin administration protocol for preventing vascular vasospasm and vessel thrombosis in free tissue transplantation.

The present invention meets this need and provides an effective, relatively long term, non-systemic neurotoxin administration, therapeutic method for preventing vascular vasospasm and vessel thrombosis in free tissue transplantation.

### DETAILED DESCRIPTION OF THE PREFERRED EMBODIMENT

The present invention is an effective, long lasting, non-systemic botulinum toxin administration protocol for preventing vascular vasospasm and vessel thrombosis in free tissue transplantation. The botulinum toxin administration protocol is herein described in the context of free tissue transfer or a “free flap” procedure, the terms free flap and free tissue transfer being used herein as synonymous terms describing the movement of tissue from one site on the body to another.

The free flap procedure initially entails a first step of dissecting a flap via incision. The blood supply including at least one vein and one artery is dissected. The vein and artery (vascular pedicle) are divided, separating the flap from the rest of the body. The flap is freed from the surrounding tissue.

In a second preparation step, the area the flap to be transferred is prepared by identifying a recipient artery and vein to which the free flap's vascular vein and artery (vascular pedicle) can be attached.

In a third botulinum pretreatment step that is optional but preferred, the recipient artery and vein are pretreated with a single local injection of botulinum toxin as described below in more detail.

In a fourth microsurgery step, the free flap is brought up to the prepared area, and the vein and artery from the flap (vascular pedicle) are anastomosed (connected) to the vein and artery identified in the wound. The anastomosis is done using a microscope, hence it is termed “microsurgery”

In a fifth suturing step, the free flap is sutured to the prepared area, and the donor site is sutured closed.

In a sixth local administration and monitoring step, a relatively long term, non-systemic local administration neurotoxin administration protocol is effected in conjunction with a periodic monitoring program to ensure the blood vessels remain patent (i.e. the vessels have good blood flow). It is this sixth step, alone or combined with the third (optional) botulinum pretreatment, that can proactively prevent vascular vasospasm and vessel thrombosis after the free tissue transplantation. As used herein “local administration” means direct injection of a neurotoxin into the recipient vessel perivascular space. Systemic routes of administration, such as oral and intravenous routes of administration, are excluded from the scope of “local administration” of a neurotoxin.

The neurotoxin is preferably a botulinum toxin, and the protocol including species, amount and frequency of the botulinum toxin administered according to the present method may vary somewhat within the scope of the disclosed invention, depending on the reason for the free flap procedure and other various patient variables including size, weight, age, and responsiveness to therapy. A presently preferred protocol is a local injection of between 1-30 units of a botulinum toxin type A (such as Botox™) administered to the injection site (i.e. into the recipient vessel perivascular space), per patient treatment session, with the patient undergoing monthly treatment sessions over a total timeframe of between two months to six months. If desired, the neurotoxin can be administered in a broader range of between about 1 and about 35 U/kg. 35 U/kg is an upper limit because it approaches a lethal dose of botulinum toxin type A. Botulinum toxin A shows an onset between 3 to 5 days and lasts several weeks to months. Muscle functional recovery occurs by a mechanism of sprouting of new nerve terminals.

As an alternative to botulinum toxin type A, one of the one of the other botulinum toxin serotypes B, C₁, D, E, F or G can be used, although not as effectively and therefore not the preferred. For example, if botulinum toxin type B is used in between 40-2000 units of botulinum toxin type B are administered to the injection site, per patient treatment session, over the same total time frame. Botulinum toxin type B can be safely administered at several orders of magnitude higher dosage, such as up to about 2,000 U/kg.

The neurotoxin can be made by a Clostridial bacterium, such as by a Clostridium botulinum, Clostridium butyricum, Clostridium beratti or Clostridium tetani bacterium. Additionally, the neurotoxin can be a modified neurotoxin, that is a neurotoxin that has at least one of its amino acids deleted, modified or replaced, as compared to the native or wild type neurotoxin. Furthermore, the neurotoxin can be a recombinant produced neurotoxin or a derivative or fragment thereof.

When administered in accordance with the foregoing protocol, systematically and non-systemically, the above-described therapeutic method helps prevent vascular vasospasm and vessel thrombosis in free tissue transplantation (free flap) procedures.

The mechanism for this result is primarily a combination of two factors: 1) the botulinum toxin A blocks sympathetically mediated cold-induced vasoconstriction by preventing recruitment of alpha 2 receptors in vascular smooth muscle; 2) Botulinum toxin A binds irreversibly to block the release of acetylcholine at the neuromuscular junction of cholinergic neurons resulting in muscle paralysis. The mechanism may also rely on secondary factors. Botulinum toxin A also prevents the release of substance P, glutamate, and it increases the release of calcitonin gene-related peptide (CGRP). CGRP is a potent vasodilator and is found to participate in the maintenance of inflammation and vasoregulation. Botulinum toxin A has also been shown to prevent the release of norepinephrine at the neuromuscular junction and subsequently prevented sympathetic vasoconstriction of vascular smooth muscle. Vasoconstriction is also dependent upon the recruitment of specific alpha 2 receptors (alpha 2c) involving a phospholipase D pathway that is completely blocked by botulinum toxin.

### Example 1

A blinded, vasospasm animal model was designed to determine the ability of Botox to prevent anastomotic thrombosis. A blinded, vasospasm animal model was designed to determine the ability of botulinum toxin A to prevent anastomotic thrombosis. Study design, animal care, and monitoring were approved by the IACUC animal care committee. Ten anesthetized Charles River CD (Sprague-Dawley-derived) rats were pretreated with a botulinum toxin A. All procedures were performed under aseptic techniques using sterile instruments.

Animals were injected with 10 units of botulinum toxin A (Botox, Allergan Inc., Irvine, Calif.) diluted in 0.5 cc of 0.9% sodium chloride injection to the perivascular space surrounding randomized femoral vessels. As an internal control the contralateral femoral vessels received normal saline. Side of injection, left versus right, was randomized and blinded to injector and surgeon at all times throughout the study. Five days post-treatment, the animals underwent femoral vessel measurements to determine effect neuromuscular blockade on vessel diameter. Using a nose cone, the animals were anesthetized with 3-5% Isofluorane initially and maintained at 1-3% until the procedure was completed. Operative sites were shaved and disinfected with alcohol (70%). Homeostasis was monitored by thermometer and maintained during surgery by a surgical light as a heat source. Digital photographs of vessels were taken with a high resolution camera (8 megapixel) and were enlarged to a size of 10×12 inch prints and then measured in relation to 1×1 mm green squared background for standardization. This resulted in an effective 50× magnification. This allowed for reproducible and reliable measurements to the 100^{th} of a millimeter. Measurement was of the external diameter of the vessel.

The Fanua-Wilgis technique was used to expose the femoral vessels. This technique involves marking the skin in the groin region in the shape of a “C.” The incision follows the mark, exposing the epigastric fat pad and the pyramidalis muscle. Dissection proceeds by retracting the skin and epigastric fat pad, exposing the femoral neuromuscular bundle. Vessels were then sectioned and reanastomosed by a single surgeon. Vessels were resutured using an Ethicon 10-0 nylon suture. Anastomosis of groin sides was randomized.

Upon completion of both femoral arteries and veins in a single rat, the blinded surgeon recorded the side deemed to be more technically difficult. Ease of anastomosis was defined by a single surgeon in terms of subjective speed, vessel quality, and effort with suturing. Anastomosis was timed from application to release of clamps. Incisions were closed with 3-0 chromic catgut mattress sutures in one layer. Following suturing, animals were subjected to a systemic treatment with a peripheral vasoconstrictor, phenylephrine (0.1 mg/kg) injected submuscularly, and a lower extremity cold challenge in an ice bath (16° C.) for 5 minutes. Vessel patency was recorded at time 0 (before cold challenge), and 1 hour after. Vessel patency was determined by reopening the incision site at one hour, and visually inspected under a microscope. Color of extremity was also noted.

All test treatments were generally well tolerated. During pretreatment with saline, one animal sustained a femoral vein puncture which was treated with pressure and otherwise uneventful. One animal expired second to anesthetic complications prior to vasospasm challenge.

A sample size of 20 vessels gives a greater than 80% chance (power) of detecting an absolute difference of 40% in vessel patency at the 0.05 probability level using McNemar's test for paired categorical data. Paired t tests were used for continuous data.

The results indicate that the therapeutic method is quite effective at preventing vascular vasospasm and vessel thrombosis in free tissue transplantation (free flap) procedures. Vessel diameter was consistently larger in all sites pretreated with botulinum toxin A. The botulinum toxin A treated arterial average diameter of 0.68+/−0.2 mm (mean+/−SEM) was significantly larger (p<0.001 by paired t test) than the matched control saline arterial average of 0.53+/−0.02 mm. The botulinum toxin A treated venous average diameter of 0.95+/−0.05 mm was significantly larger (p=0.006 by paired t test) than the matched control saline venous average of 0.73+/−0.05 mm. Moreover, ease of anastomosis and time of suturing were significantly less in the pretreated botulinum toxin A group. The botulinum toxin A treated arterial average of 22.0 minutes+/−2.5 minutes (mean+/−SD) was significantly shorter (p=0.005 by paired t test) than the matched control saline arterial average of 23.6+/−2.9 minutes. The botulinum toxin A treated venous average of 20.3+/−2.4 minutes was significantly shorter (p=0.02 by paired t test) than the matched control saline venous average of 22.2+/−3.2 minutes. The Botulinum toxin A treated femoral vessels were deemed the easier side in 60% of the animals. The other remaining 40% of femoral vessels were determined to be equivalent in ease of anastomosis.

Vasospastic challenge resulted in 89% of animals thrombosing either an artery, a vein, or both. Of saline pretreated femoral arteries, 44% thrombosed following vasospasm challenge. Of saline pretreated femoral veins, 67% thrombosed following vasospasm challenge. Twenty-two percent of the animals had both a saline pretreated artery and vein thrombosis. Eleven percent of the animals had neither a saline pretreated artery or vein thrombose. Patency was maintained in 100% of botulinum toxin A treated vessels and 44% of saline treated vessels at 1 hour (p=0.004, by McNemar's Test).

In conclusion, many factors contribute to the success of an anastomosis and the survival of a free flap. Within skilled hands, free flaps carry a very high success rate, but the most common cause for failure is vessel thrombosis. Pretreatment of blood vessels at the site of a planned micorvascular anastomosis demonstrated statistically significant enlargement of arteries and veins, decreased the difficulty of anastomosis and the time required for anastomosis. The stress challenge used in this study (cold bath and systemic administration of a peripheral vasoconstrictor) mimics the hypothesized mechanism of action of vasospasm. Pretreatment with botulinum toxin A was associated with a decreased thrombosis rate within this model. Pretreatment with botulinum toxin A had the added benefit of producing significantly larger vessels than saline pretreatment. Previous studies have demonstrated increased muscle perfusion after botulinum toxin A treatment, and this study may demonstrate a mechanism contributing to that observed phenomenon. Botulinum toxin A administration is generally safe and well tolerated with the most common adverse effect being the extension of the effect outside the intended area. This is usually a result of diffusion with larger dilutional doses. This is a rare consequence and within this study there were no observed gross effects (e.g. limb dysfunction) of systemic response to botulinum toxin A. To be certain of the location of vessels, superficial vessels such as the radial artery and vein or the superficial temporal artery and vein would be preferable and are areas for future study.

The ability for botulinum toxin A to decrease vasospasm and thrombosis may have applications for improving free flap survival in select patients. For example, previous studies have demonstrated the deleterious effects of smoking on subsequent microsurgical anastomosis. Patients particularly prone to vasospasm or with risk factors for thrombosis (e.g. smoking, diabetes, renal failure) may be good candidates for this procedure. Further studies to examine the effect of botulinum toxin treatment on other known vasoconstricting stressors would be useful.

Having now fully set forth the preferred embodiment and certain modifications of the concept underlying the present invention, various other embodiments as well as certain variations and modifications of the embodiments herein shown and described will obviously occur to those skilled in the art upon becoming familiar with said underlying concept. It is to be understood, therefore, that the invention may be practiced otherwise than as specifically set forth in the appended claims.

I claim:

1. A method for prevention of vascular vasospasm and thrombosis by local administration of botulinum toxin to free tissue transfer recipient vessels, comprising the steps of: local administration of a therapeutically effective amount of a botulinum toxin to the recipient vessels of a free tissue transplant patient, wherein the botulinum toxin is administered in an amount between 1 unit and 20,000 units, thereby preventing vasospasm and decreasing the incidence of vascular thrombosis and free tissue loss between two months and six months.

2. The method of claim 1, wherein the botulinum toxin is locally administered in an amount of between about 1 U/kg and about 35 U/kg.

3. The method of claim 1, wherein the botulinum toxin is made by a Clostridial bacterium.

4. The method of claim 1, wherein the botulinum toxin is selected from the group consisting of botulinum toxin types A, B, C, D, E, F and G.

5. The method of claim 1, wherein the botulinum toxin is botulinum toxin type A.

6. The method of claim 1, wherein the botulinum toxin is administered to the free tissue transfer recipient vessels of a patient by placement of a botulinum toxin implant on or near the recipient vessels.

7. The method of claim 1, wherein the botulinum toxin is botulinum toxin type A and the botulinum toxin administered to the free tissue recipient vessels of the patient in an amount between 1 unit and about 100 units.

8. A procedure for free tissue transplantation in a human, comprising:
a first step of dissecting a free flap of skin from a donor site via incision, said free flap including at least dissected one vein and at least one dissected artery, and separating the dissected free flap from surrounding tissue;
a second preparation step of identifying an anastamosis site having a recipient artery and vein to which the free flap vein and artery can be attached;
a third pretreatment step comprising pre-treating the anastamosis site by a single local injection of botulinum toxin proximate the recipient artery and vein;
a fourth microsurgery step comprising anastomosing the free flap vein and artery to the identified recipient artery and vein using a microscope;
a fifth suturing step including suturing the free flap to the anastamosis site, and suturing the donor site closed;
a sixth post-treatment step comprising delivering a long term, non-systemic local administration of botulinum toxin by periodic direct injection into the recipient vessel perivascular space.

9. The procedure for free tissue transplantation in a human according to claim 8, further comprising a seventh step of periodically monitoring the free flap of skin to ensure that its blood vessels remain patent.

10. The procedure for free tissue transplantation in a human according to claim 8, wherein said sixth post-treatment step comprises delivering a plurality of patient treatment sessions each including a local injection of between 1-30 units of botulinum toxin type A to the recipient vessel perivascular space.

11. The procedure for free tissue transplantation in a human according to claim 10, wherein said plurality of patient treatment sessions are delivered monthly over a total timeframe of between two months to six months.

12. The procedure for free tissue transplantation in a human according to claim 8, wherein said sixth post-treatment step comprises delivering a plurality of patient treatment sessions each including a local injection of between 40-2000 units of botulinum toxin type B to the recipient vessel perivascular space.

13. The procedure for free tissue transplantation in a human according to claim 12, wherein said plurality of patient treatment sessions are delivered monthly over a total timeframe of between two months to six months.

14. A method for preventing vascular vasospasm and vessel thrombosis after a free tissue transplantation, comprising the steps of delivering a plurality of local injections of botulinum toxin into a recipient vessel perivascular space at pre-determined time intervals.

15. The method for preventing vascular vasospasm and vessel thrombosis after a free tissue transplantation according to claim 14, wherein each local injection comprises between 1-30 units of botulinum toxin type A.

16. The method for preventing vascular vasospasm and vessel thrombosis according to claim 14, wherein said pre-determined time intervals are monthly over a total timeframe of between two months to six months.

17. The method for preventing vascular vasospasm and vessel thrombosis according to claim 14, wherein each local injection comprises between 40-2000 units of botulinum toxin type B.

18. The method for preventing vascular vasospasm and vessel thrombosis according to claim 17, wherein said pre-determined time intervals are monthly over a total timeframe of between two months to six months.
